(19) United States
(12) Patent Application Publication
(10) Pub. No.: US 2006/0159647 A1
(43) Pub. Date: Jul. 20, 2006

(54) Compositions for hygienic hand disinfection and disinfectant handwashing

(76) Inventors: Wolfgang Beilfuss, Hamburg (DE); Sabine Behrends, Appen (DE); Peter Goroncy-Bermes, Hamburg (DE); Burghard Puchstein, Hamburg (DE)

Correspondence Address: Linda K. Russell, Suite 1800, 2700 Post Oak Blvd., Houston, TX, 77056, US

(21) Appl. No.: 11/333,376

(22) Filed: Jan. 17, 2006

(30) Foreign Application Priority Data
Jan. 19, 2005 (DE) 10 2005 002 643.5

(51) Int. Cl. A61K 8/49; A61K 8/73

(52) U.S. Cl. 424/70.13; 424/70.27

(57) ABSTRACT

The invention relates to a disinfectant composition which includes a) one or more 1- or 2-(C₁- to C₂₄-alkyl)glycerol ethers, b) one or more bispyridiniumalkanes and c) one or more amine oxides, and to the use of the composition for hygienic hand disinfection and for disinfectant handwashing.

This application claims the benefit of priority under 35 U.S.C. § 119 (a) and (b) to German Application No.10 2005 002 643.5, filed Jan. 19, 2005, the entire contents of which are incorporated herein by reference.

### BACKGROUND

The present invention relates to disinfectant compositions which are preferably employed for hygienic hand disinfection and disinfectant handwashing.

The aim of hand disinfection, hand decontamination and skin antisepsis is to prevent transmission of microorganisms and viruses or to suppress their unwanted introduction into endangered regions of the body or more sensitive regions. Compositions for hygienic hand disinfection and for disinfectant handwashing must satisfy certain efficacy requirements, some of which are defined in standards. Various methods are possible for treating the hands after contamination.

Hygienic hand disinfection complying with EN 1500 as rubbing method without addition of water causes the death or inactivation of the transient microorganisms on the hands, without any risk of microbes being disseminated in the surroundings and without any risk of recontamination of the hands by microorganisms possibly present in the water. Disinfectant handwashing complying with EN 1499 with a microbicidal composition using tap water is likewise directed against transient microorganisms without precluding their dissemination in the surroundings. It serves in particular to reduce microbes during the washing procedure, but cannot replace hand disinfection. In disinfectant handwashing, the composition is rubbed in undiluted and foamed with a little water, and the hands are cleaned and thoroughly rinsed with water.

Compositions for disinfectant handwashing and hygienic hand disinfection must be effective after acting for short times (e.g. 30 seconds or 1 minute). In the efficacy tests, it is important that there is a good effect (RF value >3 orders of magnitude) after acting for these short times. For toxicological reasons it is additionally necessary for, besides adequate efficacy, in particular the compatibility with human skin to be ensured.

Commercially available compositions for disinfectant handwashing are usually alcohol- or surfactant-containing liquid soaps and wash lotions ready for use with further biocidal agents added. Known compositions comprise as biocidal agents for example short-chain alcohols and as excipients refatting agents, moisturizers and fragrances to improve the skin compatibility and acceptance. To enhance the efficacy and to achieve a residual effect intended to prevent the number of microbes on the hands increasing again, the known compositions often also comprise additional agents such as biguanides (e.g. chlorhexidine), quaternary ammonium compounds (e.g. benzalkonium chloride), phenol derivatives (e.g. ortho-phenylphenol) or carboxylic acids.

U.S. Pat. No. 4,542,125 discloses combinations of octenidine dihydrochloride with (higher alkyl) (di-lower-alkyl)amine oxide as dip compositions for disinfecting the teats of cows. U.S. Pat. No. 4,420,484 and DE 27 08 331 A1 describe antimicrobial skin-cleansing compositions which comprise octenidine dihydrochloride and optionally amine oxide.

DE 102 05 883 A1 relates to an aqueous antiseptic based on bispyridiniumalkanes. Besides octenidine dihydrochloride, the antiseptic comprises a nonionic surfactant selected from alcohol polyalkoxylates, polysorbates and alkyl glycosides. The further presence of amine oxide is emphasized as disadvantageous. DE 196 47 692 A1 relates to an aqueous disinfectant solution which comprises octenidine dihydrochloride, alkyl alcohol, nonionic and/or cationic surfactant and skin-compatible α-hydroxy carboxylic acid. The presence of amine oxides is referred to as disadvantageous.

In addition, an aqueous composition which includes octenidine dihydrochloride, cocamidopropylamine oxide, glyceryl cocoate, glycerol, hydroxyethylcellulose, lactic acid and allantoin is known as commercial product.

The known compositions for disinfectant handwashing have a number of disadvantages. Thus, some products show an efficacy which is not always satisfactory, or the desired efficacy is achieved only after acting for a lengthy time. Some compositions additionally have insufficient skin compatibility. Thus, compositions based on chlorhexidine are reported to be prone to skin incompatibility, it being suggested for example that there is partial release of chloroaniline.

An additional factor is that agents with organically bound halogen such as chlorhexidine have only conditional environmental compatibility. Chlorhexidine is moreover sufficiently effective only with a comparatively high concentration of the agent (e.g. 2% by weight) in wash products and may lead to discolorations on contaminated surfaces. Poly(hexamethylenebiguanide) hydrochloride is a polymeric biguanide salt whose structure is not exactly defined. For this reason, no medicinal product with this agent yet has marketing authorization in Germany. The polymeric biguanide may be employed only in a concentration of up to 0.3% by weight in cosmetic compositions.

Hence there is a need for compositions with improved efficacy and, at the same time, good skin compatibility.

It has now surprisingly been found that the problems of the prior art can be overcome by a composition which includes a) one or more 1- or 2-(C₁- to C₂₄-alkyl) glycerol ethers (glycerol monoalkyl ethers), b) one or more bispyridiniumalkanes and c) one or more amine oxides. The compositions according to the invention have an excellent effect with, at the same time, good skin compatibility and stability. They show a distinctly improved efficacy compared with known compositions. Moreover, synergistic increases in effect are observed in some cases. The possible inhibition of the efficacy of bispyridiniumalkanes, which is observed with numerous surfactants, does not occur with amine oxides.

The compositions can be employed as products for personal hygiene and as medical wash products, high-value, soap-free wash products for all hand, skin and body washing and as bath additive, and for disinfection or decontamination of animate (e.g. skin, hands, mucous membrane, wounds) and inanimate surfaces (e.g. apparatuses, instruments, endoscopes).

a) Glycerol Monoalkyl Ethers

It is possible in principle according to the invention to use all suitable glycerol monoalkyl ethers. Cycloalkyl groups are also intended as alkyl groups. The alkyl group of the glycerol monoalkyl ether is preferably an aliphatic, branched or unbranched C₃- to C₁₈-alkyl group, more preferably a branched or unbranched C₆- to C₁₂-hydrocarbon group, in particular a branched or unbranched octyl or dodecyl group. Examples of glycerol monoalkyl ethers employed according to the invention are dodecyl glycerol ether, decyl glycerol ether, octyl glycerol ether, propyl glycerol ether, octadecyl glycerol ether (batyl alcohol), hexadecyl glycerol ether (chimyl alcohol) and octadecenyl glycerol ether (selachyl alcohol). 1-Glycerol monoalkyl ethers are preferably employed. 1-(2-Ethylhexyl)-glycerol ether (Sensiva® SC 50) and 1-dodecylglycerol ether are particularly preferred. In addition, 3-(1-menthoxy)-1,2-propanediol (glycerol monomenthyl ether) can be employed.

The content of glycerol monoalkyl ether is generally in the range from 0.05 to 5% by weight, preferably 0.1 to 3% by weight, more preferably 0.2 to 2% by weight, such as 0.5 to 1 % by weight.

b) Bispyridiniumalkane

All suitable bispyridiniumalkanes can in principle be used according to the invention. The bispyridiniumalkane employed according to the invention is preferably a bis[4-(substituted amino)-1-pyridinium]alkane of the general formula I or II

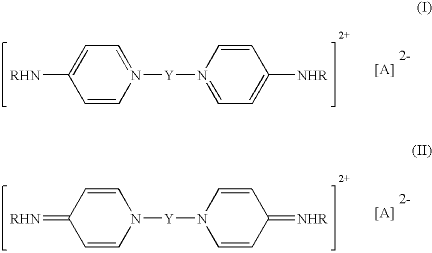

where Y is an alkylene group having 4 to 18 carbon atoms, R is an alkyl group having 6 to 18 carbon atoms or a cycloalkyl group having 5 to 7 carbon atoms or the phenyl radical which may be substituted by a halogen atom, and A is an anion or a plurality of anions. In a particularly preferred embodiment, the bispyridiniumalkane employed according to the invention is N,N′-(1,10-decanediyldi-1[4H]-pyridinyl-4-ylidene)bis(1-octanamine) dihydrochloride (octenidine dihydrochloride). Octenidine disulphate is also suitable moreover.

In a preferred embodiment, the content of bispyridiniumalkane is in the range from 0.01 to 2% by weight, more preferably 0.05 to 1.5% by weight, in particular 0.1 to 1% by weight, such as 0.2 to 0.5% by weight.

c) Amine Oxide

All suitable amine oxides can in principle be employed according to the invention. Amine oxides, which are N-oxides of tertiary amines, include aliphatic amine oxides, cyclic amine oxides (such as n-alkylmorpholine oxide) and aromatic amine oxides (such as pyridine N-oxides). In a preferred embodiment, the amine oxide has the general formula
R¹R²R³N—O
in which R¹ is methyl, ethyl or 2-hydroxyethyl, R² is methyl, ethyl or 2-hydroxyethyl, R¹ and R² together may be morpholine, R³ is alkyl having 8 to 18 carbon atoms or R⁴CONH(CH₂)ₙ where R⁴ is alkyl having 8 to 18 carbon atoms, and n is in the range from 1 to 10, preferably 1 to 5, more preferably 2 to 4, and is in particular 3, and 2-hydroxyethyl may be condensed with from 1 to 2000 ethylene oxide, ethylene oxide/propylene oxide or propylene oxide units.

Examples of amine oxides are cocamidopropylamine oxide, N-cocomorpholine oxide, decyldimethylamine oxide, dimethylcetylamine oxide, dimethylcocamine oxide, dimethyl-hydr. tallow-amine oxide, dimethyllaurylamine oxide, dimethylmyristylamine oxide, (2-hydroxyethyl)cocamine oxide and oleamine oxide. See also “International Cosmetic Ingredient Dictionary and Handbook”, 10th edition 2004, volume 3, pages 2268-2275 (Surfactants-Cleansing Agents). The risk of nitrosamine formation can be offset by suitable protective measures, e.g. during production.

In a preferred embodiment, the amine oxide is cocamidopropylamine oxide, i.e., R⁴CO is the acyl radical derived from the fatty acids of coconut oil, n=3, and R¹ and R² are methyl.

In a preferred embodiment, the content of amine oxide is in the range from 1 to 20% by weight, more preferably 2 to 15% by weight, in particular 6 to 13% by weight, such as 8 to 12% by weight, for example about 10% by weight.

Preference is moreover given to compositions which additionally include from 1 to 5% by weight, preferably 2 to 3% by weight, refatting agent, such as polyol fatty acid ester, e.g. glycerol ester, preferably glycerol cocoate, isopropyl myristate, isopropyl palmitate, and triglycerides.

Compositions which are additionally preferred are those additionally including from 0.05 to 0.5% by weight, more preferably 0.1 to 0.3% by weight, of skincare additive such as allantoin, glycerol or sodium gluconate.

A composition of the invention may additionally comprise from 0.1 to 5% by weight, preferably 0.5 to 2% by weight, of thickener, especially hydroxyethylcellulose, hydroxymethylcellulose and hydroxypropylcellulose.

To adjust the pH of the composition of the invention it is additionally possible for from 0.1 to 4% by weight, preferably 0.4 to 2% by weight, more preferably 0.6 to 1% by weight carboxylic acid to be present in the composition of the invention, such as hydroxy carboxylic acid, for example lactic acid, succinic acid, maleic acid, tartaric acid, citric acid or mandelic acid, with particular preference for lactic acid.

The composition of the invention is preferably in the form of an aqueous composition and comprises from 10 to 99% by weight, more preferably 30 to 98% by weight, in particular 50 to 95% by weight, such as 60 to 90% by weight, 65 to 88% by weight, 70 to 86% by weight or 80 to 85% by weight, based on the composition.

A particularly preferred composition includes:
a) 0.5 to 1 % by weight, such as about 0.75% by weight, 1-(2-ethylhexyl)glycerol ether,
b) 0.2 to 0.5% by weight, such as about 0.3% by weight, octenidine dihydrochloride and
c) 8 to 12% by weight, such as about 10.0% by weight, cocamidopropylamine oxide,
   and, where appropriate
d) 0.8 to 1.5% by weight, such as about 1.1% by weight, glycerol,
e) 2 to 3% by weight, such as about 2.5% by weight, glycerol cocoate,
f) 0.1 to 0.3% by weight, such as about 0.2% by weight, allantoin,
g) 0.5 to 2% by weight, such as about 1.0% by weight, hydroxyethylcellulose,
h) 0.6 to 1% by weight, such as about 0.8%, lactic acid and/or
i) 80 to 85% by weight, such as about 83.15% by weight, water.

The composition may additionally comprise functional additives such as colorants, perfume, buffers, electrolytes and moisturizing factors. The preferred pH of the compositions is from 4 to 8, preferably 4 to 6.

The invention further relates to the use of the compositions for hygienic hand disinfection or for disinfectant handwashing. The compositions according to the invention are suitable for hygienic and surgical hand disinfection, hygienic handwashing, hand decontamination, skin decontamination, mucous membrane decontamination, personal hygiene washing lotion, as antimicrobial washing lotion, for (whole) body washing and care in connection with MRSA (methicillin-resistant Staphylococcus aureus) and furthermore for disinfectant handwashing, for hygienic catheter care in patients, as handwashing product such as, for example, as antimicrobial soap, handwashing gel or handwashing lotion. They can be employed advantageously in all sectors with enhanced hygiene requirements in the medical and nonmedical sector, e.g. hospitals, medical practices, old people's and nursing homes, and in the food products and kitchen sectors.

Compositions according to the invention may also be formulated as alcoholic hand disinfectants, gel compositions, ointment compositions and antimicrobial coatings.

It was surprising that an effect is achieved after only a short time with compositions according to the invention containing glycerol monoalkyl ether for hand disinfection. Although glycerol monoalkyl ethers are known, for example from DE 42 40 674 C1, to act as deodorant agents, disinfection is immaterial for deodorants. For this reason, known deodorant agents merely have antimicrobial activity, whereas hand disinfectants must have microbicidal activity. An additional factor is that glycerol monoalkyl ethers on their own have virtually no microbicidal effect, and accordingly are unsuitable as (sole) agents for hand disinfection. It was therefore surprising that glycerol monoalkyl ethers contribute in the compositions according to the invention to the hand-disinfectant effect.

In addition, deodorant compositions which are intended by their nature to remain on the skin for a lengthy period are preferably free of surfactants. Thus, compatibility between deodorant agents and surfactants is immaterial in deodorant compositions. By contrast, it was surprising that compositions according to the invention can be formulated with up to 20% by weight amine oxide without the efficacy of the bispyridiniumalkanes and glycerol monoalkyl ethers being impaired.

The compositions according to the invention additionally have the following advantages:
Good compatibility in combination with good antimicrobial efficacy and excellent short-term effect (acting for a time of, for example, 30 or 60 seconds), and very good effect on Gram-negative bacteria.
Good efficacy for viruses (enveloped and non-enveloped) and multidrug-resistant microorganisms such as MRSA.
No need to add a further preservative because they are self-preservative.
The solubility in water and water-based compositions of glycerol monoalkyl ethers (such as 1-(2-ethylhexyl)glycerol ether) is only limited (solubility in water 0.1% by weight). The presence of bispyridiniumalkane in the compositions according to the invention leads to a significantly improved solubility in water of the glycerol monoalkyl ether.

The advantages of the invention are evident in particular from the following examples.

### EXAMPLES

The following compositions were formulated. All data are in % by weight.

| | | B |
|---|---|---|
| | A | (comparative) |
| Polybiguanide | — | — |
| Sensiva ® SC 50 | 0.75 | — |
| Octenidine dihydrochloride | 0.30 | 0.30 |
| Fatty acid amidopropyldimethylamine oxide | 10.00 | 10.00 |
| Glycerol | 1.10 | 1.10 |
| Allantoin | 0.20 | 0.20 |
| Glycerol cocoate | 2.50 | 2.50 |
| Hydroxyethylcellulose | 1.00 | 1.00 |
| Lactic acid | 0.81 | 0.81 |
| Alkyl polyglycoside | — | — |
| PEG 150 distearate | — | — |
| Deionized water | ad 100 | ad 100 |

¹ Amount not known.
Method for Determining the Efficacy of the Compositions

The efficacy of the compositions of the invention was tested in the quantitative suspension test. Compare standard methods of the DGHM for testing chemical disinfection methods, J. Gebel, H.-P. Werner, A. Kirsch-Altena, K. Bansemir, mhp Verlag GmbH, Wiesbaden, Germany, Method 9.1 (date 1 Sep. 2001) (quantitative suspension test with bacteria (apart from mycobacteria and fungi).

| S. aureus | A | B (comparative) |
|---|---|---|
| 15″ | 1.83 | — |
| 30″ | 3.81 | 0 |
| 1′ | 4.50 | 2.38 |
| 3′ | not determined | 3.52 |
| 5′ | not determined | 4.28 |
| E. hirae | A | B (comparative) |
| 15″ | 0 | not determined |
| 30″ | 3.03 | 0 |
| 1′ | 3.94 | 3.61 |

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. Disinfectant composition which includes:
a) one or more 1- or 2-(C₁- to C₂₄-alkyl)glycerol ethers (glycerol monoalkyl ethers);
b) one or more bispyridiniumalkane; and
c) one or more amine oxides.

2. Composition according to claim 1, characterized in that the alkyl group of the glycerol monoalkyl ether is a branched or unbranched C₃- to C₁₈-alkyl group, in particular a 2-ethylhexyl group or a dodecyl group.

3. Composition according to claim 1, characterized in that the glycerol monoalkyl ether is 1-(2-ethylhexyl)glycerol ether.

4. Composition according to claim 1, characterized in that it includes from 0.05 to 5% by weight glycerol monoalkyl ether.

5. Composition according to claim 1, characterized in that the bispyridiniumalkane is bis[4-(substituted amino)-1-pyridinium]alkane of the general formula I or II 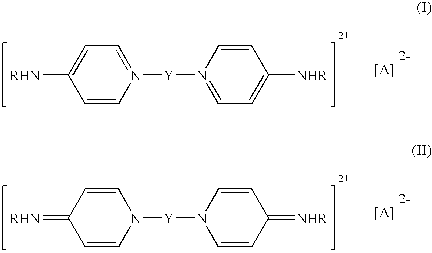
where Y is an alkylene group having 4 to 18 carbon atoms, R is an alkyl group having 6 to 18 carbon atoms or a cycloalkyl group having 5 to 7 carbon atoms or the phenyl radical which may be substituted by a halogen atom, and A is an anion or a plurality of anions.

6. Composition according to claim 1, characterized in that the bispyridiniumalkane is octenidine dihydrochloride.

7. Composition according to claim 1, characterized in that it includes from 0.01 to 2% by weight bispyridinium alkane.

8. Composition according to claim 1, characterized in that the amine oxide has the general formula
R¹R²R³N—O
in which R¹ is methyl, ethyl or 2-hydroxyethyl, R² is methyl, ethyl or 2-hydroxyethyl, R¹ and R² together may be morpholine, R³ is alkyl having 8 to 18 carbon atoms or R⁴CONH(CH₂)ₙ where R⁴ is alkyl having 8 to 18 carbon atoms, and n is in the range from 1 to 10, and 2-hydroxyethyl may be condensed with from 1 to 2000 ethylene oxide, ethylene oxide/propylene oxide or propylene oxide units.

9. Composition according to claim 1, characterized in that the amine oxide is cocamidopropylamine oxide.

10. Composition according to claim 1, characterized in that it includes from 6 to 13% by weight amine oxide.

11. Composition according to claim 1, characterized in that it additionally includes from 0.5 to 5% by weight glycerol.

12. Composition according to claim 1, characterized in that it additionally includes from 1 to 5% by weight refatting agent, preferably glycerol cocoate.

13. Composition according to claim 1, characterized in that it additionally includes from 0.05 to 0.5% by weight skincare additive, preferably allantoin.

14. Composition according to claim 1, characterized in that it additionally includes from 0.1 to 5% by weight thickener, preferably hydroxyethylcellulose.

15. Composition according to claim 1, characterized in that it additionally includes from 0.1 to 4% by weight hydroxy carboxylic acid, preferably lactic acid, succinic acid, maleic acid, tartaric acid, citric acid or mandelic acid.

16. Composition according to claim 1, characterized in that it additionally includes from 60 to 90% by weight water.

17. Composition according to claim 1, characterized in that includes:
a) 0.5 to 1% by weight 1-(2-ethylhexyl)glycerol ether;
b) 0.2 to 0.5% by weight octenidine dihydrochloride; and
c) 8 to 12% by weight cocamidopropylamine oxide,
and, where appropriate,
d) 0.8 to 1.5% by weight glycerol;
e) 2 to 3% by weight glycerol cocoate;
f) 0.1 to 0.3% by weight allantoin;
g) 0.5 to 2% by weight hydroxyethylcellulose;
h) 0.6 to 1% by weight lactic acid; and/or
i) 80 to 85% by weight water.

18. Use of a composition which includes:
a) one or more 1- or 2-(C₁- to C₂₄-alkyl)glycerol ethers (glycerol monoalkyl ethers);
b) one or more bispyridiniumalkanes; and
c) one or more amine oxides,
for hygienic hand disinfection or for disinfectant handwashing.
